(19) United States
(12) Patent Application Publication
(10) Pub. No.: US 2024/0424214 A1
(43) Pub. Date: Dec. 26, 2024

(54) DRUG DELIVERY DEVICE

(71) Applicant: ACTUATE TECHNOLOGY LTD, Horsham (GB)

(72) Inventor: John Palmer-Felgate, Horsham (GB)

(21) Appl. No.: 18/699,458

(22) PCT Filed: Oct. 7, 2022
(86) PCT No.: PCT/GB2022/052551

§ 371 Date: Apr. 8, 2024

(30) Foreign Application Priority Data
Oct. 8, 2021 (GB) 2114436.5
Jun. 15, 2022 (GB) 2208764.7

(51) Int. Cl. A61M 5/20; A61M 5/315; A61M 5/32

(52) U.S. Cl. CPC A61M 5/2033; A61M 5/31511; A61M 5/3257

(57) ABSTRACT

A drug delivery device is disclosed in which an activation element is arranged to prevent lateral movement of a syringe plunger until the drug delivery device is activated. Upon activation of the drug delivery device, the activation element is displaced to thereby permit lateral movement of the plunger under the influence of a drive element.

### BACKGROUND

Drug delivery devices, such as autoinjectors, injector pens and patch pumps, can be used to automate the delivery of injected drugs.

Drug delivery devices are available in a variety of sizes and designs, and at least part of each device is generally disposed of once one or more doses of the drug have been delivered. This “single-use” nature of drug delivery devices results in the generation of a significant amount of waste, which is generally handled as biomedical sharps waste and therefore incinerated.

In addition, existing drug delivery devices often have complex activation mechanisms that are expensive to manufacture and complicated to assemble. For example, US 2016/0199588 A1 discloses an autoinjector having a plunger activation mechanism in which the plunger is released by rotating a plunger boss out of a bayonet slot. During assembly, the plunger is inserted into a case and rotated so that the boss on the plunger engages the bayonet slot.

There is a need for drug delivery devices with fewer components and/or components that are made from more environmentally friendly materials in order to reduce the environmental burden of these devices. However, some environmentally friendly materials (such as biopolymers) are not as strong as conventional materials (such as normal polymers) and lack the necessary long-term strength to contain the relatively high forces exerted by components such as drive springs used in some drug delivery devices.

The aim of the present invention is to provide a drug delivery device that is reliable, simple to assemble, and can be made from environmentally friendly materials.

### SUMMARY OF THE INVENTION

According to a first aspect of the invention, there is provided a drug delivery device comprising: a housing; a syringe received within the housing and comprising a plunger, wherein the plunger comprises a first guide surface arranged to abut a second guide surface within the housing at a non-perpendicular angle relative to a longitudinal axis of the plunger; a drive element arranged to exert a depression force upon the plunger; and, an activation element movable between a first (or initial) position in which the activation element is arranged to prevent lateral movement of the plunger within the housing and a second (or activated) position in which the activation element is arranged to allow lateral movement of at least part of the plunger within the housing, wherein when the activation element is displaced from the first position to the second position, abutment between the first guide surface and the second guide surface causes a lateral displacement of the at least part of the plunger due to the depression force of the drive element, thereby releasing the plunger to move from an undepressed position to a depressed position under the depression force of the drive element.

Many existing drug delivery devices rely on small flexible plastic components that are typically thin and long sections of plastic for locking the plunger rod into position. These sections are under constant load during storage prior to use and are a vulnerable part of the design. By controlling lateral movement of the plunger (rather just longitudinal movement of the plunger), the activation mechanism of the present invention can retain a drive element (such as a drive spring) and release the plunger with a minimal amount of force, e.g. the force of a user pressing the device onto an injection site. This means that the activation element does not need to be made of an especially strong material, unlike the components of conventional drug delivery device activation mechanisms. Also, this latch mechanism requires just two rigid interlocking components.

The angled (i.e. non-perpendicular and non-parallel relative to the longitudinal axis) abutment between the first and the second guide surfaces results in a normal reaction force that has both a longitudinal component that opposes the depression force of the drive element (which acts in a direction parallel to the longitudinal axis of the plunger) and a lateral force that urges the plunger towards the activation element. This allows for the resulting component of the drive element force acting upon the activation element to be near to zero, thereby allowing the activation element to move with minimal resistance.

Another benefit of the activation mechanism of the first aspect is that it does not require any rotation of the plunger. This means that the device can be assembled in a linear fashion (i.e. without having to rotate any components during assembly).

The depression force is a biasing force that urges the plunger from an undepressed/withdrawn/retracted position into a depressed position (the plunger may initially be partially or fully retracted, and the depression force may act to urge the plunger towards a more depressed position). The depression force acts (substantially) in the longitudinal direction (i.e. along the longitudinal axis of the plunger), although there may be relatively small lateral components of the depression force too.

The longitudinal axis of the plunger is the axis of the plunger that is parallel to the direction in which the plunger moves when it is depressed into the barrel of the syringe (i.e. parallel to the depression force provided by the drive element). The longitudinal axis of the plunger is substantially the same as the longitudinal axis of a barrel of the syringe.

The lateral direction is the direction perpendicular to the longitudinal axis of the plunger. Lateral displacement means displacement with a non-negligible component in the lateral direction.

The syringe may be a pre-filled syringe, for example, and may be any suitable size. Alternatively, the syringe could be a cartridge with a septum or any other drug container having a plunger. The tip of the syringe may optionally comprise a needle for delivering the drug into a patient. Alternatively, the tip of the syringe may be connected or connectable to a tube or similar.

The second guide surface may optionally be on an interior surface of the housing or on a frame or other component received within the housing.

The first guide surface may optionally be on a first guide element (such as a fin/lug) protruding radially/laterally from the plunger, and the second guide surface may be on a second laterally protruding guide element within the housing (e.g. on the interior surface of the housing or on a frame or other component received within the housing).

Alternatively, the first guide surface may be on a guide element (such as a fin/lug) protruding radially/laterally from the plunger, and the second guide surface may be in a guide recess (or groove), e.g. on the interior surface of the housing or on a frame or other component received within the housing. The guide recess may be shaped to receive the laterally protruding guide element.

Alternatively, the first guide surface may be on a guide recess (or groove) in the plunger, and the second guide surface may be on a laterally protruding guide element, e.g. on the interior surface of the housing or on a frame or other component received within the housing. The guide recess may be shaped to receive the laterally protruding guide element.

The first guide surface may optionally be angled (i.e. non-perpendicular and non-parallel) relative to the longitudinal axis of the plunger. Additionally/alternatively, the second guide surface may be angled (i.e. non-perpendicular and non-parallel) relative to the longitudinal axis of the plunger. Preferably, the angle of the first guide surface corresponds to the angle of the second guide surface.

The first and/or second guide surfaces may be flat, or one or both may be curved/non-planar.

Optionally, the activation element may be movable in a longitudinal direction within the housing. That is, movement of the activation element from the first position to the second position may comprise longitudinal movement of the activation element within the housing. Additionally or alternatively, movement of the activation element from the first position to the second position may comprise rotational movement and/or movement in a direction tangential to an outer surface of the plunger.

The activation element may be an activation plate.

Optionally, the activation element may comprise an aperture or window shaped to receive a laterally protruding locking element of the plunger (e.g. a laterally protruding fin or lug) when the activation element is in the second position, and the laterally protruding locking element may abut the activation element in the first position.

Alternatively, the plunger may comprise a locking recess (or groove) arranged to receive a laterally protruding locking element of the activation element when the activation element is in the second position, and the laterally protruding lock element may abut the plunger in the first position.

The drive element can be any component suitable for applying a longitudinal depression force on the plunger.

Preferably, the drive element is a compression spring.

The drug delivery device may optionally further comprise a flexible guide tube within or around the compression spring. The guide tube prevents the compression spring kinking or buckling within the housing when the piston is depressed but allows the plunger to move laterally (unlike the metal pins used on existing designs).

Alternatively, the drive element may be a piston. The piston may optionally be damped and/or resettable.

Optionally, the drug delivery device may further comprise a moveable needle guard arranged to shield a needle of the syringe. The needle guard may optionally comprise the activation element.

The lateral displacement of the plunger may comprise bending and/or pivoting of plunger. For example, the plunger may pivot around a tip of the plunger received within the barrel of the syringe.

The drug delivery device could be an autoinjector, an injector pen, a patch pump or any other drug delivery device having a syringe driven by a drive element.

According to a second aspect of the invention, there is provided a autoinjector cap comprising: a cap housing; and, a gripping plate received in an opening in a side of the cap housing and arranged to grip a rigid needle shield (RNS) of a syringe retained within the autoinjector, wherein the gripping plate comprises an aperture comprising a pair of opposing gripping surfaces configured to engage opposing sides of an outer surface of the RNS; and, wherein the gripping plate is bent along a line bisecting the aperture such that each of the opposing gripping surfaces is angled towards a tip of the autoinjector cap.

The side of the housing extends between a first end of the cap shaped to receive the RNS and a second end of the cap opposing the first end. The tip of the autoinjector cap is at the second end of the cap.

Conventional autoinjector caps use gripping elements such as metal tubes and star washers to grip the RNS surface. However, these caps often require a high force to attach them, and the teeth of star washers are prone to bending/inverting in an “over-centre” fashion when the cap is removed, resulting in the cap being removed without the RNS (i.e. a failure of the cap).

In contrast, having gripping surfaces on opposing sides of a plate which is bent along a line bisecting an aperture allows the RNS of the pre-filled syringe to be inserted into the cap with relatively little force (because the gripping surfaces are angled toward the tip of the autoinjector cap) and with virtually no risk of the gripping plate inverting.

Another benefit compared to conventional autoinjector caps is that the gripping plate can be made of a relatively thin/weak material. While gripping elements such as star washers rely upon the stiffness of the metal to prevent unwanted bending of the teeth, the bent shape of the gripping plate of the present invention means that the gripping surfaces are naturally angled to grip the RNS during removal of the cap without the need for bent protruding teeth.

The gripping plate of the present invention can also be used to grip different diameters of RNS by adjusting the angle of the bend, thereby reducing inventory overheads and allowing for larger manufacturing tolerances. In contrast, conventional gripping mechanisms (such as star washers) are generally only suitable for a single RNS diameter and need to be manufactured with high precision.

Preferably, the gripping surfaces engage the outer surface of the RNS at a non-perpendicular angle relative to a longitudinal axis of the RNS. This non-perpendicular engagement allows the cap to be pushed onto the RNS with a relatively low force while providing a high gripping force on the outer surface of the RNS.

Preferably, the gripping surfaces are positioned on opposing sides of a perimeter of the aperture.

The gripping plate is preferably formed from a resilient material and may optionally be retained in the opening under strain (e.g. under compression) by abutment between the gripping plate and an inner surface of the opening. Under strain means that the resilient gripping plate is displaced from its equilibrium position such that a resilient biasing force urges it towards its original position. Retaining the gripping plate in the opening under strain allows the gripping plate to be held in the opening by the friction created between the gripping plate and the inner surface of the opening, which reduces the risk of the gripping plate falling out of the opening during sub-assembly shipping and final assembly of the autoinjector.

Optionally, the gripping plate may have an L-shaped profile. The gripping plate may be bent at an angle of (about) 90 degrees (i.e. a right-angle), or it may have a larger or smaller angle. The arms of the L-shape may be (approximately) the same length or they may be different lengths.

Alternatively, the gripping plate may have a W-shaped profile. Having a W-shaped profile allows the opening in the side of the cap housing to be larger, which makes the cap housing easier to manufacture and makes the cap easier to assemble.

Preferably, the gripping plate is bent along the line bisecting the aperture at an angle of between 110 degrees and 150 degrees, more preferably between 120 degrees and 140 degrees, even more preferably between 125 and 135 degrees, and most preferably (about) 130 degrees. Being bent at these angles means that the RNS can be easily inserted into the cap but also ensures sufficient grip between the RNS and the gripping plate.

Preferably, at least part of the opening has a profile that is angled to match a profile of the gripping element. Having a matching profile means that the opening supports the gripping surfaces during removal of the cap, which prevents the gripping element and/or gripping surfaces inverting and losing grip on the RNS.

The cap housing may optionally and advantageously be formed as a single moulded component.

According to a third aspect of the invention, there is provided an autoinjector comprising the cap of the second aspect.

The autoinjector of the third aspect may optionally have any of the features of the first aspect of the invention, although these features are not essential.

According to a fourth aspect of the invention, there is provided an autoinjector comprising: a housing; a needle guard moveable between a retracted position and an extended position in which the needle guard is arranged to shield a needle of a syringe retained within the housing; and, a bias spring arranged to bias the needle guard into the extended position, wherein the needle guard comprises a first latch element arranged to interface with a corresponding second latch element fixed within the housing; wherein the autoinjector further comprises an activation element initially in a first position in which the activation element is positioned to obstruct the second latch element and thereby prevent abutment between the first latch element and the second latch element; and, wherein when the needle guard is moved from the extended position to the retracted position, abutment between the needle guard and the activation element causes the activation element to move from the first position to a second position in which the second latch element is exposed to the first latch element such that subsequent movement of the needle guard from the extended position to the retracted position is prevented by abutment between the first latch element and the second latch element.

In the extended position, part of the needle guard at least partially surrounds the tip of the needle. In the retracted position, the tip of the needle is exposed (i.e. is not surrounded by part of the needle guard).

Compared to existing designs, the needle guard activation mechanism of the fourth aspect requires fewer components and therefore helps to reduce waste. In addition, in existing designs, the needle guard is generally in a different position when the cap is on compared to the position that activates the autoinjector (i.e. there are multiple retracted positions), and the “lockout” position of the needle guard is different to the “cap off” position (i.e. there are also multiple extended positions). In contrast, the invention of the fourth aspect can use a single fully extended position and a single fully retracted position.

One issue with the multiple extended/retracted positions on existing devices is that trigger positions are often very near to activation positions, which can lead to accidental activation e.g. when the device is dropped. To overcome this, existing designs use complex lockout mechanisms, which increases the number of components required in the device compared to the activation mechanism of the fourth aspect.

In addition, the activation mechanism of the fourth aspect can be activated by stored energy in metal springs only rather than relying on the spring force from a bent plastic component. Plastic features do not offer long term spring forces as the plastic will creep and mould to its stored position, so the activation mechanism of the fourth aspects provides an autoinjector with improved longevity.

Optionally, the autoinjector may further comprise a removable cap, wherein the needle guard is initially in the retracted position and is configured to move from the retracted position into the extended position under the influence of the bias spring upon removal of the removable cap.

In existing designs, the needle guard is generally maintained in the extended position before the cap is removed. In contrast, the activation mechanism of the fourth aspect allows the cap to be applied while the needle guard is in the retracted position, which makes it easier for the cap to grip the RNS of the needle because more of the RNS is exposed (this is especially beneficial when the autoinjector of the fourth aspect is used in combination with the autoinjector cap of the second aspect). This can be achieved by inserting the first latch element into the housing of the autoinjector adjacent to (i.e. alongside) the activation element during assembly so that the first latch element does not engage the activation element until the needle guard is extended upon removal of the cap.

Preferably, the needle guard can articulate without flexing to thereby allow the first latch element to engage with the second latch element.

The activation element may be the same activation element as in the first aspect of the invention, i.e. movement of the activation element from the first position to the second position may also activate a syringe plunger of the autoinjector. Alternatively, the activation element may be a separate component. The activation element may optionally be an activation plate.

Preferably, the first latch element is a longitudinal arm of the needle guard.

Optionally, the bias spring may be further arranged to provide a lateral force upon the longitudinal arm to bias the longitudinal arm towards the second latch element. This allows for improved engagement between the first and second latch elements, which reduces the risk of accidental retraction of the needle guard after the device has been used.

Preferably, the second latch element is on an inner surface of the housing. Alternatively, the second latch element may be on another element within the housing that is fixed relative to the syringe, e.g. on a support/frame or similar.

For example, the second latch element may be a recess or protrusion within the housing.

The autoinjector for the fourth aspect may have any features of the first and/or second aspects, although these features are not essential.

According to a fifth aspect of the invention, there is provided a syringe retention member for retaining a syringe in a drug delivery device, comprising: an annular collar; and, a pair of opposing arms extending longitudinally from a distal side of the annular collar, each of the pair of opposing arms pivotable relative to the collar about a respective pivot point, wherein each of the pair of opposing arms comprises a shoulder section distal of the respective pivot point such that the pair of opposing arms together provide a syringe abutment shoulder for engaging in a circumferential gap between a barrel of the syringe and a rigid needle shield (RNS) of the syringe.

According to a sixth aspect of the invention, there is provided a syringe retention member for retaining a syringe in a drug delivery device, comprising: an annular collar; and, one or more support arms extending longitudinally from a distal side of the annular collar, each of the one or more support arms pivotable relative to the collar about a respective pivot point, wherein each of the one or more support arms comprises a shoulder section distal of the respective pivot point that provides a syringe abutment shoulder for engaging in a circumferential gap between a barrel of the syringe and a rigid needle shield (RNS) of the syringe.

In existing drug delivery devices, a syringe carrier and front-end component are generally assembled during final assembly where the syringe has to be assembled into the carrier before being pushed into the front sub-assembly. In the syringe retention member of the fifth and sixth aspects, the need for a separate syringe carrier is eliminated by the way in which the arms pivot: during assembly, the arms are manually articulated apart to allow the insertion of the syringe (i.e. the syringe cannot be inserted between the arms without the arms being held apart).

Having the shoulder section (and therefore the syringe abutment shoulder) distal of the pivot point creates a self-locking effect that acts upon the syringe: when a load is placed upon the syringe plunger, the force acting on the syringe abutment shoulder causes the arms to grip the syringe more tightly rather than prising them apart. Compared to existing designs (e.g. those in which the pivot point is distal of the shoulder section) this means that no outer collar is required.

As known to one skilled in the art, the tip of the syringe is positioned towards the proximal end of the drug delivery device (i.e. the end with the cap), and the distal end of the drug delivery device is the opposite end of the drug delivery device (i.e. away from the tip of the syringe and the cap of the device). The distal side of the annular collar is the side of the collar positioned towards the distal end of the drug delivery device once the drug delivery device is assembled.

Preferably, when a syringe is inserted into the syringe retention member an abutment surface of the syringe (e.g. on the outer surface of the neck of the syringe) abuts a respective engagement surface of the syringe abutment shoulder of each respective support arm, wherein the respective engagement surface is positioned proximally to a straight line extending through the respective pivot point of the respective support arm perpendicular to a tangent of the abutment surface where the respective engagement surface abuts the abutment surface (or, if the abutment surface is flat, perpendicular to the abutment surface where the respective engagement surface abuts the abutment surface). In other words, the point on the abutment surface where the respective engagement surface abuts the abutment surface is at a proximal position (i.e. closer to the needle end of the assembled autoinjector) on the tangent relative to the straight line (it would be equally valid to say that that is positioned radially inward instead of positioned proximally).

Having the engagement surface positioned proximally to the straight line extending through the pivot point perpendicular the tangent of the abutment surface ensures that the support arms pivot radially inwards when a load is placed upon the syringe plunger, thereby achieving the self-locking effect without the need for any collars or similar around the support arms.

The syringe abutment shoulder may optionally be formed by an inwardly-projecting syringe protrusion on each of the arms.

Preferably, an opening in the annular collar is shaped to receive a needle guard.

Preferably, an outer surface of each of the pair of opposing arms comprises a first locking element configured to interface with a corresponding second locking element on a housing or syringe carrier of the autoinjector so as to prevent removal of the syringe retention member from the housing. Once assembled, the housing acts as an external tube around the syringe retention member pushing the pair of opposing arms onto the syringe and preventing the syringe from moving.

Preferably, an outer surface of each of the support arms comprises a first locking element configured to interface with a corresponding second locking element on a housing or syringe carrier of the autoinjector so as to prevent removal of the syringe retention member from the housing. Once assembled, the housing acts as an external tube around the syringe retention member pushing the support arms onto the syringe and preventing the syringe from moving.

Preferably, when a syringe is received within the syringe retention member, a barrel of the syringe provides a radially outward reaction force that prevents the opposing arms from flexing inwards.

Optionally, the drug delivery device may be an autoinjector, a patch pump, or an injection pen.

The one or more support arms may comprise a pair of opposing arms. The one or more support arms may also comprise more than two arms (e.g. three arms), preferably arranged equidistantly around the distal side of the annular collar.

According to a seventh aspect of the invention, there is provided a drug delivery device comprising the syringe retention member of the fifth or sixth aspects.

The drug delivery device of the seventh aspect may optionally have any of the features of the first, second and/or fourth aspects of the invention, although these features are not essential.

According to an eighth aspect of the invention, there is provided an autoinjector cap comprising: a cap housing; and, a gripping plate received in an opening in a side of the cap housing and arranged to grip a rigid needle shield of a syringe retained within the autoinjector, wherein the gripping plate comprises an aperture comprising a pair of opposing gripping surfaces configured to engage opposing sides of an outer surface of the rigid needle shield; and, wherein the gripping plate is bent along a line bisecting the aperture such that each of the opposing gripping surfaces is angled towards a tip of the autoinjector cap.

According to a ninth aspect of the invention, there is provided an autoinjector comprising: a housing; a needle guard moveable between a retracted position and an extended position in which the needle guard is arranged to shield a needle of a syringe retained within the housing; and, a biasing element arranged to bias the needle guard into the extended position, wherein the needle guard comprises a first latch element arranged to interface with a corresponding second latch element fixed within the housing; wherein the autoinjector further comprises an activation element initially in a first position in which the activation element is positioned to obstruct the second latch element and thereby prevent abutment between the first latch element and the second latch element; and, wherein when the needle guard is moved from the extended position to the retracted position, abutment between the needle guard and the activation element causes the activation element to move from the first position to a second position in which the second latch element is exposed to the first latch element such that subsequent movement of the needle guard from the extended position to the retracted position is prevented by abutment between the first latch element and the second latch element.

### BRIEF DESCRIPTION OF THE DRAWINGS

Examples of the present invention will now be described in detail with reference to the accompanying drawings, in which:

FIG. 1 illustrates a side view of autoinjector;

FIG. 2 shows a cutaway view of the autoinjector;

FIG. 3 show an exploded view of the components of the autoinjector;

FIG. 4 illustrates a cutaway view an activation mechanism of the autoinjector;

FIGS. 5a-5d show cutaway views the autoinjector during different states of drug delivery;

FIGS. 6a-c illustrate activation of the autoinjector;

FIG. 7 is a side view of an injector pen;

FIG. 8 illustrates an autoinjector cap;

FIG. 9 shows a side view of the autoinjector cap;

FIGS. 10a-c show alternative views of the autoinjector cap;

FIGS. 11a-e illustrate a shield activation mechanism of the autoinjector;

FIG. 12 shows a side view of the shield activation mechanism;

FIGS. 13a and 13b illustrate a syringe retention member of the autoinjector;

FIGS. 14a-f illustrate how the syringe retention member is assembled with a syringe and a needle guard;

FIGS. 15a and 15b illustrate the engagement between the syringe retention member and the syringe and needle guard;

FIG. 16 shows a cutaway view of the syringe retention member;

FIG. 17 shows an alternative cutaway view of the syringe retention member; and

FIG. 18 shows a simplified illustration of the geometry of the syringe retention member.

### DETAILED DESCRIPTION

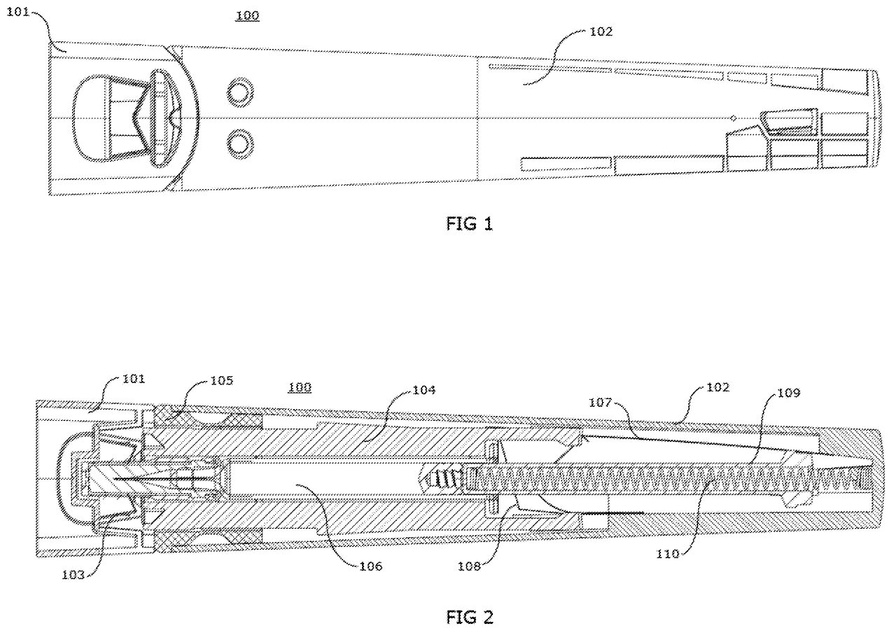

FIG. 1 shows a drug delivery device in the form of an autoinjector 100 having a cap 101 and a housing 102. As shown in FIG. 2, the cap 101 of the autoinjector 100 has a rigid needle shield (RNS) grip element 103, and the housing 102 contains a needle guard 104, a syringe retention member 105, a pre-filled syringe (PFS) 106 (pre-filled with a drug to be administered), an activation element in the form of an activation plate 107, a bias spring 108, a plunger 109 (which may also be referred to as a plunger rod) and a drive element in the form of a drive spring 110.

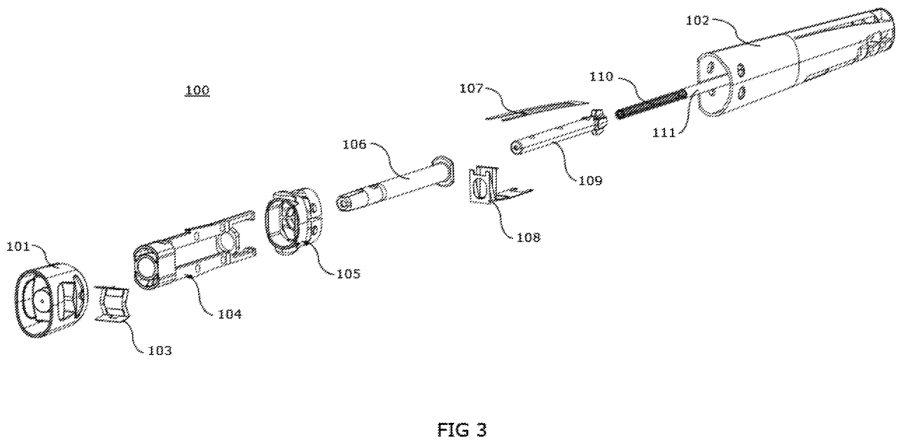

The components of the autoinjector are shown in more detail in the exploded view of FIG. 3, in which a flexible guide tube 111 is also visible.

The housing 102 may be any container or shell suitable for holding the components of the autoinjector 100, and it may be partially or fully enclosed. To allow the components of the autoinjector 100 to be checked visually, the housing 102 may be partially or fully transparent. Alternatively, the housing 102 may be translucent or opaque, for example if required for a light-sensitive drug. The housing 102 may also have one or more windows through which components of the drug delivery device are visible, which may be formed as openings or transparent sections of the housing 102.

The RNS grip element 103 acts to grip the RNS of the PFS 106 such that the RNS is removed with the cap 101 when a user separates the cap 101 from the housing 102 (e.g. immediately prior to using the autoinjector 100 to administer a drug).

The PFS 106 may be any suitable device having a barrel and a plunger. The drug stored in the PFS 106 could be in liquid form, or it may alternatively be in another form such as a powder that mixes with a liquid before injection.

The syringe retention member 105 retains the needle guard 104 and PFS 106 within the housing 102. The needle guard 104 is movable longitudinally within the housing 102. In its initial position (shown in FIGS. 1 and 2), the needle guard 104 substantially surrounds the needle of the PFS 106 and functions both to activate the autoinjector 100 when it is pressed to the skin and to prevent the needle of the PFS 106 being exposed after use.

The bias spring 108 serves a dual purpose of providing a retaining force on the PFS 106 (e.g. on a flange of the PFS 106) that prevents the PFS 106 moving back into the housing 102 during injection and of providing a restoring force on the needle guard 104. The illustrated bias spring 108 is formed of a resilient metal plate, although other mechanisms could be used instead (such as a plastic bias spring, or a pair of springs or other biasing elements acting separately on the PFS 106 and needle guard 104).

The plunger 109 is used to drive the drug out of the PFS 106 under the force of the drive spring 110. Although the illustrated drive spring 110 is a compression spring, this could be replaced with an alternative biasing element/drive element such as a piston. The flexible guide tube 111 surrounds a least part of the drive spring 110 and acts to prevent bucking of the drive spring 110 within the housing when driving the plunger 109. The flexible guide tube 111 could alternatively be positioned within the drive spring 110 or replaced by a flexible pin within the drive spring 110.

The activation plate 107 prevents movement of the plunger 109 until the drug is to be delivered.

### Activation Mechanism

The activation mechanism of the autoinjector 100 will now be described in more detail. While the activation mechanism will be primarily described in relation to an autoinjector, it should be understood that the activation mechanism could also be incorporated into other automated drug delivery devices such as patch pumps and injector pens.

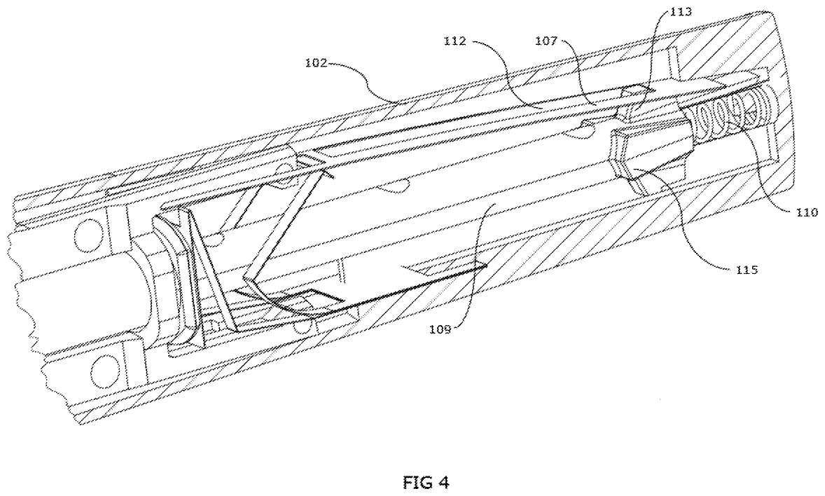

The components of the activation mechanism are shown in detail in FIG. 4. As previously mentioned, the activation plate 107 prevents movement of the plunger 109 until the drug is to be delivered. While conventional activation mechanisms oppose longitudinal movement of the plunger 109 (and therefore act as to directly oppose the compression force of the drive spring 110), the illustrated activation mechanism instead opposes lateral movement of the plunger 109 (i.e. movement of the plunger 109 in a direction perpendicular to the longitudinal extent of the plunger 109). As a consequence, the activation plate 107 only needs to exert a relatively low stopping force to prevent movement of the plunger 109. Conventional activation mechanisms often oppose the longitudinal movement by means of a flexible locking member. This member is part of the load chain through which the plunger rod is held in position. The resulting design requires both a flexible but strong member. These are often locked in place by another component which prevents the flexing member moving laterally.

As visible in FIG. 4, which shows an initial state of the autoinjector 100, the activation plate 107 initially abuts against a laterally protruding locking fin 113 (also referred to as a laterally protruding locking element) on the plunger 109. The abutment between the activation plate 107 and the locking fin 113 is in a direction substantially perpendicular to the longitudinal axis of the plunger 109 (i.e. in a lateral direction) and therefore prevents lateral movement of the plunger 109 within the housing 102.

The activation plate 107 has a window (or aperture) 112 which is shaped to receive the locking fin 113. As discussed in more detail below, when the needle guard 104 is pressed against a patient's skin, this causes the activation plate 107 to move longitudinally relative to the plunger 109 and thereby bring the window 112 into alignment with the locking fin 113 such that the locking fin 113 can move into the window 112.

While the illustrated activation plate 107 is preferably made of metal, it could also be made of another material such as plastic. In addition, the activation plate 107 could be integrated into the needle guard 104 rather than a discrete component. Furthermore, the configuration could be reversed such that the plunger comprises a window or recess shaped to receive a laterally protruding locking element on an activation element, such that movement of the activation element brings the window/recess/groove into alignment with the laterally protruding locking element.

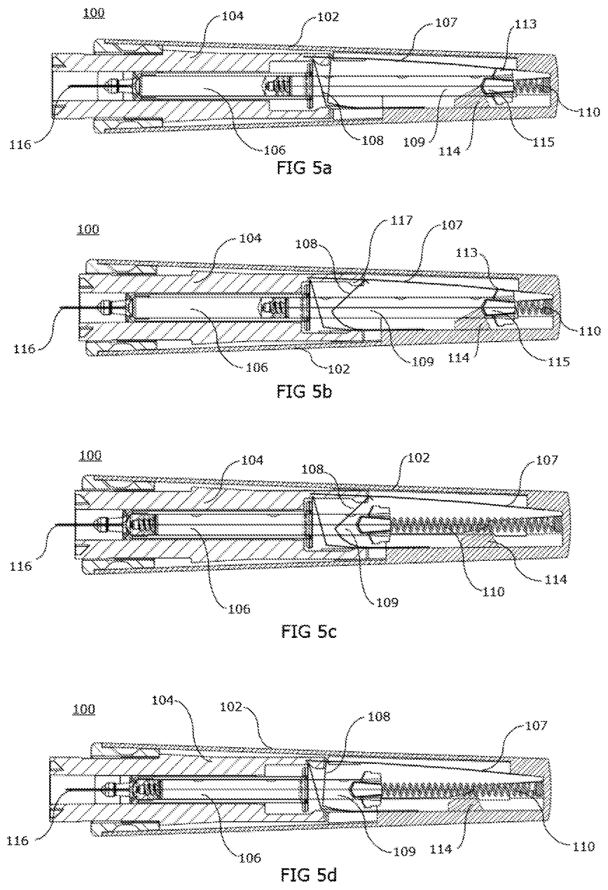

To prevent longitudinal movement of the plunger 109 until the autoinjector 100 is activated, a first guide surface on a leading edge of a laterally protruding guide fin 115 abuts against a second guide surface on a laterally protruding guide projection 114 (shown in FIGS. 5a-d) within the housing 102. Although the illustrated guide projection 114 is on the housing 102, it should be understood that the second guide surface could equally be on another element within the housing 102, such as on a frame or similar. Similarly, the configuration could be modified such that the second guide surface is on a recess within the housing, or such that the plunger comprises a guide recess shaped to receive a laterally protruding guide element within the housing 101 (e.g. on the housing or another element within the housing, such as on a frame or similar).

The first guide surface abuts the second guide surface at a non-perpendicular (and non-parallel) angle relative to the longitudinal axis of the plunger 109. This results in a normal reaction force that has both a longitudinal component that opposes the compression force of the drive spring 110 and a lateral force that urges the plunger 109 towards the activation plate 107. As discussed above, lateral abutment between the locking fin 113 and the activation plate 107 prevents lateral movement of the plunger 109 such that the plunger 109 is initially held in equilibrium under the force of the drive spring 109 in combination with the normal reaction forces between the first and second guide surfaces and between the locking fin 113 and the activation plate 107.

In the illustrated autoinjector 100, the non-perpendicular abutment angle is achieved by having an angled leading edge (first guide surface) on the guide fin 115 shaped to abut a correspondingly angled edge (second guide surface) in the guide projection 114. However, the first and second guide surfaces do not necessarily need to have corresponding angles: for example, one surface could be angled relative to the longitudinal axis of the plunger 109, whereas the other could be perpendicular to the longitudinal axis of the plunger 109 as long as the relative angled between them is non-perpendicular to the longitudinal axis of the plunger 109.

While the locking fin 113 and guide fin 115 are arranged at a distal end of the plunger 109 (i.e. away from the tip of the PFS 106), one or both of these could alternatively be positioned elsewhere along the longitudinal extend, e.g. closer to the barrel of the PFS 106. The activation plate 107 and/or guide projection 114 would have to be repositioned accordingly. Similarly, although the locking fin 113 and guide fin 115 are both fin shaped, they could instead have other shapes and be referred to more generally as a laterally protruding locking element and laterally protruding guide element respectively (and the window 112 and guide projection 114 could be shaped accordingly).

The operation of the activation mechanism will now be described in more detail with reference to FIGS. 5a-5d.

FIG. 5a shows the autoinjector 100 in an initial state (i.e. prior to activation) with the cap 101 removed. Removal of the cap 101 results in the removal of the RNS of the PFS 106, thereby exposing a needle 116 of the PFS which is surrounded by an end of the needle guard 104 in FIGS. 5a and 5d.

In this initial state, the plunger 109 is retracted from the barrel of the PFS 106, and the drive spring 110 is under compression and therefore exerts a depression force upon the plunger 109.

To activate the autoinjector 100, the needle-end of the autoinjector 100 is pressed against the patient's skin. Abutment between the needle guard 104 and the patient's skin pushes the needle guard 104 longitudinally into the housing 102 into the state shown in FIG. 5b, while the needle 116 penetrates the surface of the patient's skin. When the needle guard 104 retracts longitudinally into the housing 102, abutment between the activation plate 107 and an arm 117 of the needle guard 104 causes longitudinal movement of the activation plate 107 from an initial locking position (in which the activation plate 107 laterally abuts the locking fin 113 of the plunger 109) to an activated position (in which the window 112 of the activation plate 107 is aligned with the locking fin 113 of the plunger 109).

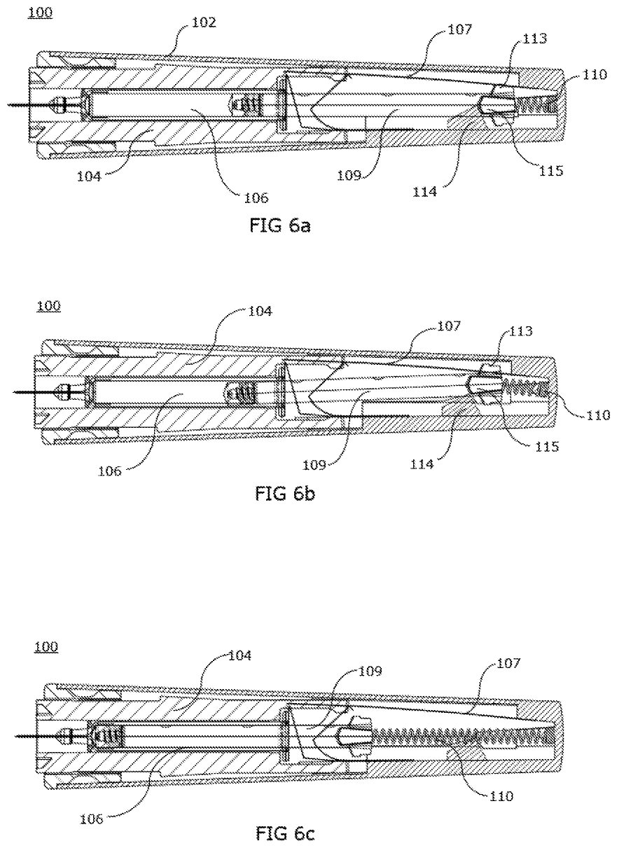

As discussed in more detail below with reference to FIGS. 6a-6c, movement of the activation plate 107 to the activated position releases the plunger 109, thereby allowing the plunger 109 to drive into the PFS 106 under the restoring force of the compression spring 110 into the position shown in FIG. 5c. This depression of the plunger 109 causes at least some of the drug in the PFS 106 to be expelled from the PFS 106 and injected into the patient via the needle 116. The plunger 109 may optionally have notches on its outer surface that interact with the bias spring 108 to provide audible feedback as the plunger 109 is depressed. In addition, there may be rails within the housing 102 that guide the plunger 109.

Once the drug has been delivered, the autoinjector 100 is removed from the patient's skin. The restoring force of the bias spring 108 then urges the end of the needle guard 104 to extend from the housing 102 as shown in FIG. 5d, thereby surrounding the needle 116. The needle guard 104 is preferably locked in this position as discussed below to prevent further use and to prevent injury due to accidental contact with the needle 116. The autoinjector 100 can then be disposed of, for example in a sharps bin.

The procedure by which the plunger 109 is released is illustrated in more detail in FIGS. 6a-6c.

FIG. 6a shows the autoinjector 100 in an instantaneous state in which the activation plate 107 has been displaced by the needle guard 104 so as to bring the window 112 of the activation plate 107 into alignment with the locking fin 113 of the plunger 109. This is the same configuration as shown in FIG. 5b.

The alignment between the window 112 and the locking fin 113 removes the lateral abutment force that was previously preventing lateral movement of the distal end of the plunger 109 (i.e. the end of the plunger 109 distal from the injection end/needle 116). As discussed above, the normal reaction force between the first guide surface (on the guide fin 115 of the plunger 109) and the second guide surface (on the guide projection 114) has a lateral component that urges the distal end of the plunger 109 towards the activation plate 107.

The movement of the activation plate 107 therefore removes the equilibrium between the plunger 109 and the activation plate 107, which causes the distal end of the plunger 109 to move laterally into the position shown in FIG. 6b as the locking fin 113 enters the window 112 in the activation plate 107. The guide fin 115 simultaneously slides against the guide projection 114, resulting in a slight longitudinal movement of the plunger 109 into the barrel of the PFS 106 in addition to the lateral movement of the plunger 109.

In FIG. 6b, the lateral movement of the plunger 109 manifests as a pivoting of the plunger 109 about the tip (proximal end) of the plunger 109, which is received within the barrel of the PFS 106. The lateral movement could alternatively be achieved by a bending or flexing of the plunger 109.

The lateral movement of the plunger 109 continues until the first guide surface disengages from the second guide surface when the guide fin 115 slides out of the guide projection 114. At this point, the lateral and longitudinal normal reaction forces arising due to the abutment between the first guide surface and the second guide surface are removed, and the plunger 109 is free to move under the force of the drive spring 110 alone, which acts longitudinally to depress the plunger 109 into the barrel of the PFS 106, thereby expelling the drug from the PFS 106 though the needle 116 as described above in relation to FIGS. 5a-5d.

The illustrated autoinjector 100 has no coaxial components other than the drive spring 110 and plunger 109, which allows for inspection of the internal mechanism after assembly (e.g. though a transparent housing 102) and also allows the user to view progress during injection as the plunger 109 can be seen moving to its final position without being obscured by the needle guard 104.

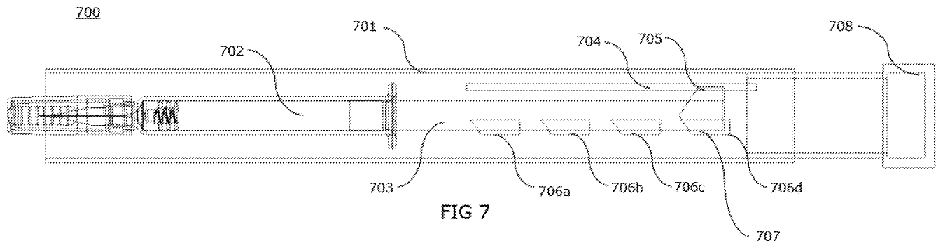

As mentioned above, the activation mechanism could be integrated into other drug delivery devices such as patch pumps and injector pens. An exemplary injector pen 700 is shown in FIG. 7. The injector pen has a housing 701, a PFS 702, a plunger 703, an activation plate 704 having a plurality of windows (not visible), a laterally protruding locking fin 705, a plurality of guide recesses 706a-d, a laterally protruding guide fin 707 and an activation button 708. Alternative arrangements are envisaged in which the window is an open ‘U’ shape.

The injector pen 700 is activated when the user actuates the activation button 708, which brings a window on the activation plate 704 into alignment with the locking fin 705 in the same manner described above for the autoinjector. Having a plurality of windows and a plurality of guide recesses 706a-d means that the injector pen 700 can be used to provide multiple doses of a drug, with each dose being administered using the same activation mechanism. The guide fin 707 will be received in each guide recess sequentially and can only move to the next guide recess upon activation of the injector pen 700.

It is envisaged that the autoinjector 100 described above could also be modified to have multiple windows on the activation plate 107 and/or multiple guide recesses so that multiple doses of a drug can be administered in the same manner as the injector pen 700 in FIG. 7.

While the illustrated activation plates 107 and 704 move longitudinally within the housings 102 and 701 respectively, the activation plates could instead be replaced by other activation elements (such as tubes around the outside of the plungers) that move e.g. tangentially relative to the surface of the plungers or in any other direction, and/or that rotate within the housing so as to move from a first position in which the activation element prevents lateral movement of the plunger to a second position in which lateral movement of the plunger is allowed. What is important is that the activation element initially provides a force that prevents lateral movement of the plunger in a lateral direction and, upon activation, moves to a second (activated) position in which the plunger can move laterally.

### Autoinjector Cap

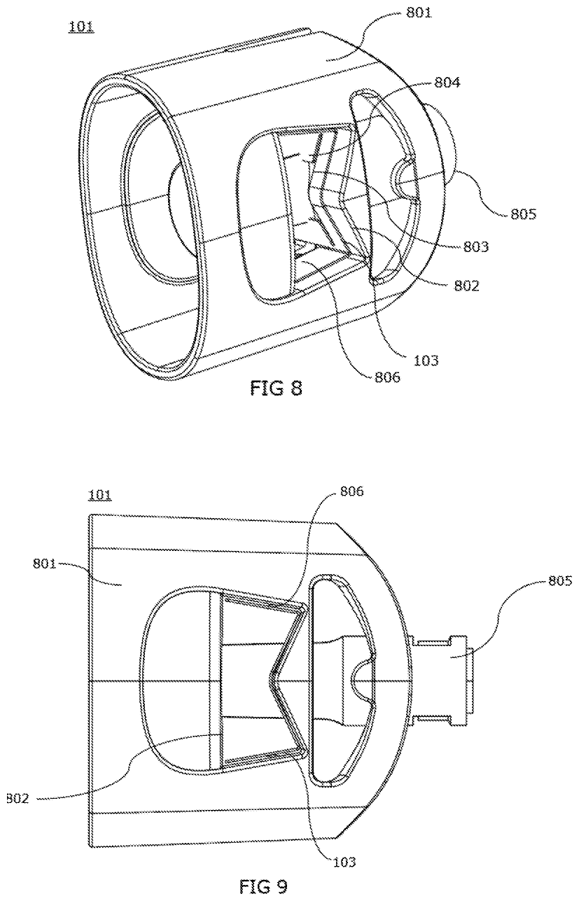

FIG. 8 shows the cap 101 of the autoinjector 100 in more detail. As mentioned above, the cap 101 has a grip element 103 which acts the grip the RNS of the PFS 106 to ensure that the RNS is removed from the PFS 106 when the cap 101 is separated from the housing 102.

The cap 101 itself comprises a cap housing 801 having an opening 802 in its side shaped to receive the grip element 103. The gripping element 103 is formed as a plate having an aperture 803 and may alternatively be referred to as a gripping plate. The gripping element 103 is bent along a line that approximately bisects the aperture 803 (i.e. a line that is positioned approximately centrally between gripping surfaces 804 described below). The angle formed by the bend can vary depending upon the size and configuration of the cap 101, but it is less than 180 degrees (no bend at all) and greater than 0 degrees (completely bent). Preferably, the angle of the bend is about 130 degrees.

The gripping surfaces 804 are positioned on opposing sides of the perimeter of the aperture 803 (i.e. symmetrically about the line bisecting the aperture 803). The illustrated gripping surfaces 804 are formed as projections extending from the perimeter of the aperture 803, although non-projecting surfaces are also envisaged.

The gripping surfaces 804 are positioned to grip the RNS 805 of the PFS 106 on opposing sides of the RNS 805. The illustrated RNS 805 features raised ridges which improve the engagement between the gripping surfaces 804 and the RNS 805, but these are optional features and the gripping surfaces 804 are also capable of gripping a smooth RNS (i.e. without raised ridges).

The illustrated gripping element 103 is also bent on opposing sides of the aperture 803 to form a W-shape with wings 806 on either side. The W-shaped profile of the gripping element 103 allows the opening 802 to be larger, which makes the cap housing 801 easier to manufacture and makes the cap 101 easier to assemble. However, the gripping element 103 could alternatively be formed with fewer or more bends, such as a single bend bisecting the aperture 803 (e.g. with an L-shaped profile or similar).

A side view of the cap 101 is shown in FIG. 9. The opening 802 has a similar bent/angled profile to the gripping element 103 (i.e. at least part of the opening 802 has a profile that matches the profile of the gripping element 103), which facilitates insertion of the gripping element 103 into the opening 802 and also provides structural support to the gripping element 103 during removal of the cap 101. In the illustrated example, the tips of the wings 806 of the gripping element 103 abut against an internal surface of the opening 802, thereby preventing longitudinal movement of the gripping element 103 relative to the cap 101.

The gripping element 103 is preferably made of a resilient material and may optionally be inserted into the opening 802 under strain (e.g. under compression with the wings 806 pressed towards each other) such that the resilient restoring force of the gripping element results in abutment between the gripping element 103 and the inner surface of the opening 802, thereby retaining the gripping element 103 within the opening 802 by friction. This allows the gripping element 103 to be inserted into to cap 101 prior to full assembly of the autoinjector 100 without risk of the gripping element falling out of the opening 802.

The bent nature of the gripping element 103 results in the gripping surfaces 804 being angled towards the tip of the autoinjector cap (i.e. away from the end of the cap that receives the RNS 805). The angled gripping surfaces 804 engage the outer surface of the RNS at a non-perpendicular angle relative to the longitudinal axis of the RNS and therefore allow the RNS 805 to be inserted into the cap 101 with relatively low force whilst providing an extremely strong gripping force on the outer surface of the 805, which mitigates the risk of the cap 101 separating from the RNS 805 when the cap is removed from the autoinjector 100.

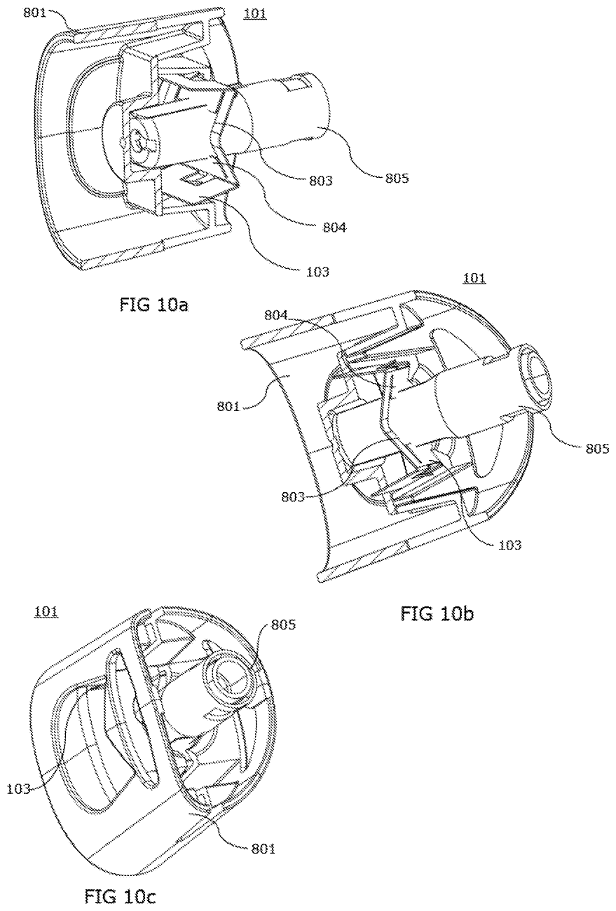

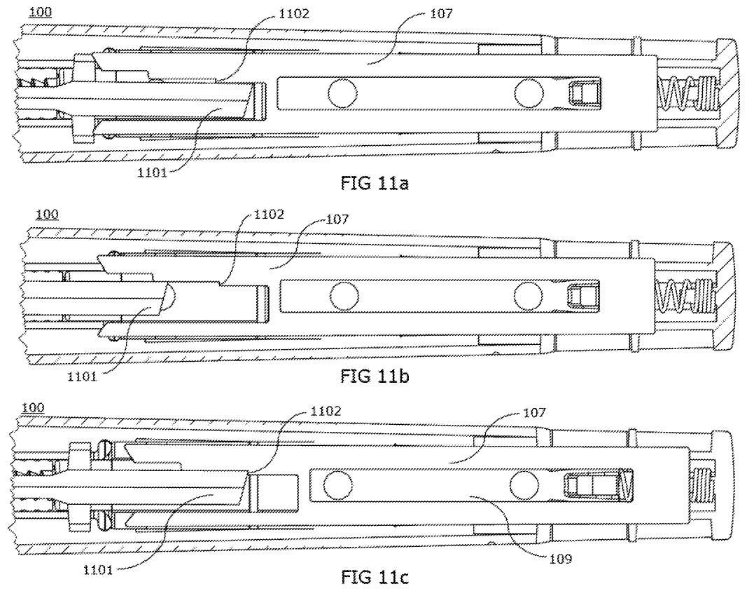

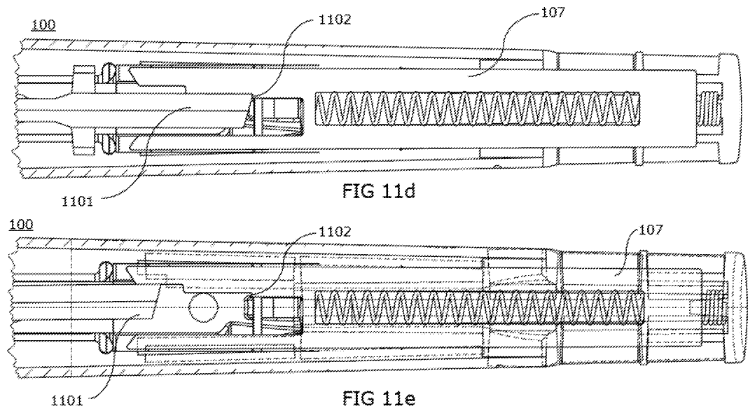

Alternative cutaway views of the cap 101 are shown in FIGS. 10a-c.

### Activation Logic

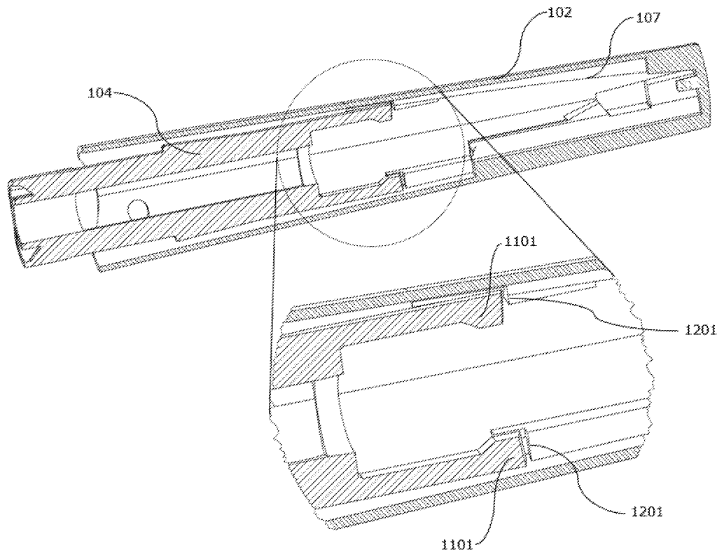

FIGS. 11a-e illustrate how the activation plate 107 can also be used to control the position of the needle guard 104 to prevent insertion (into the skin) or exposure of the needle 116 after the device has been used.

FIG. 11a shows the autoinjector 100 in an initial configuration, e.g. with the cap 101 attached. The needle guard 104 is held in a retracted position by the cap 101. The illustrated activation plate 107 has a stepped slot that allows a needle guard arm 1101 of the needle guard 104 to be inserted beyond the step 1102 during assembly of the autoinjector 100. As visible in FIG. 11a, the needle guard arm 1101 is initially bent in to a non-equilibrium position by abutment against the activation plate 107. Alternative arrangements are envisaged in which the needle arm 1101 is inserted alongside the activation plate 107 (or another activation element) rather than into a stepped slot.

When the cap 101 is removed, the needle guard 104 moves to an extended position under the force of the bias spring 108 as shown in FIG. 11b, and the resilience of the needle guard arm 1101 brings the needle guard arm 1101 into lateral alignment with the step.

As shown in FIG. 11c, when the needle guard 104 is subsequently retracted (e.g. when the autoinjector 100 is pressed against a user's skin), the end of the needle guard arm 1101 abuts against the step 1102. This abutment between the needle guard arm 1101 and the step 1102 causes a longitudinal displacement of the activation plate 107, which exposes a latch element 1201 (visible in FIG. 12) on the inner surface of the housing.

The displacement of the activation plate 107 also triggers activation of the plunger 109 as discussed above and shown in FIG. 11d.

When the autoinjector 100 is removed from the user's skin, the needle guard 104 is again extended under the force of the bias spring 108 into the position shown in FIG. 11e. Further retraction of the needle guard 104 into the retracted position is then prevented by abutment between the needle guard arm 1101 (which acts as a first latch element) and the latch element 1201 within the housing (which acts as a second latch element), i.e. the needle guard 104 is locked in the fully extended position.

The bias spring 108 also provides a laterally outward force upon the needle guard arm 1101, which acts to guide the needle guard arm 1101 into the latch element 1201 and thereby ensure that the needle guard arm 1101 engages with the latch element 1201.

Although not essential, the second needle guard arm 1101 may also engage with another latch element 1201 on the opposite side of the housing 102 as shown in FIG. 12. The engagement between the second needle guard arm 1101 and the other latch element 1201 may also be assisted by the bias spring 108 as described above.

While the latch element 1201 illustrated in FIG. 12 is in the form of a protrusion that abuts against the needle guard arm 1101, the latch element 1201 could also be replaced by e.g. a recess that serves the same function as the protruding latch element 1201 (i.e. to abut against the needle guard arm).

Although the illustrated activation plate 107 serves the dual purpose of activating the plunger 109 and controlling the position of the needle guard 104, the activation plate 107 could be replaced with one of more activation elements that each serve only one of these functions, i.e. a single activation element for activating the plunger 109 and/or a single activation element for controlling the position of the needle guard 104.

### Syringe Retention Member

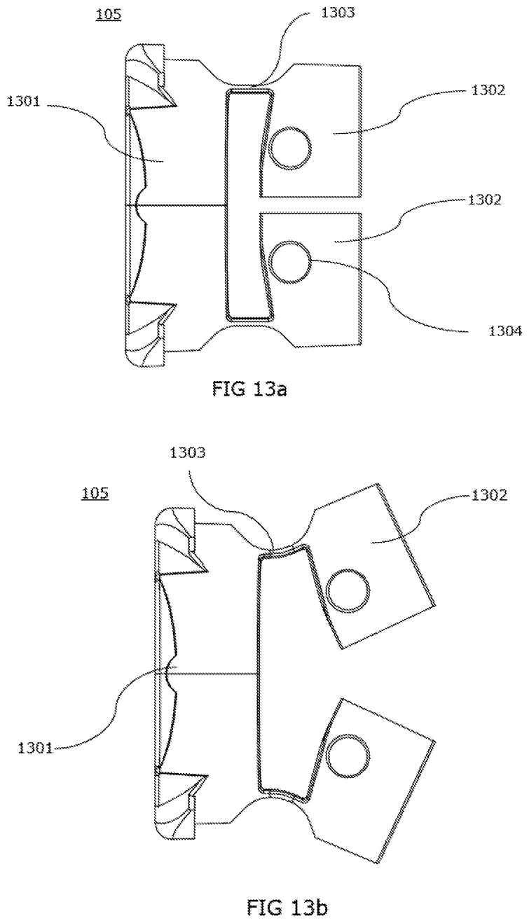

The syringe retention member 105 is shown in more detail in FIGS. 13a and 13b. The syringe retention member 105 has an annular collar 1301 with a pair of opposing arms 1302 (also referred to as support arms) extending longitudinally from a distal side of the collar (i.e. the side of the collar distal from the needle of the syringe when the autoinjector 100 is assembled). While the illustrated embodiment has two support arms, it should be understood that alternative embodiments are envisaged with three or more arms, preferably in which the arms are arranged equidistantly around the distal side of the annular collar 1301. Alternatively, embodiments are also envisaged with a single support arm. Such embodiments may optionally further comprise a static (i.e. non pivoting) support element on an opposing side of the collar to the single support arm.

Each opposing arm 1302 is pivotable/articulatable about a pivot point 1303, which is formed as a narrow section of each opposing arm 1302 in the illustrated syringe retention member 105. The outer surface of the syringe retention member 105 features a plurality of locking elements 1304 (on the opposing arms 1302) arranged to interface with corresponding locking elements on an inner surface of the housing 102. For example, the locking elements 1304 on the syringe retention member 105 could be a plurality of protrusions/bosses shaped to interface with a corresponding plurality of detents on the housing 102 (or vice-versa).

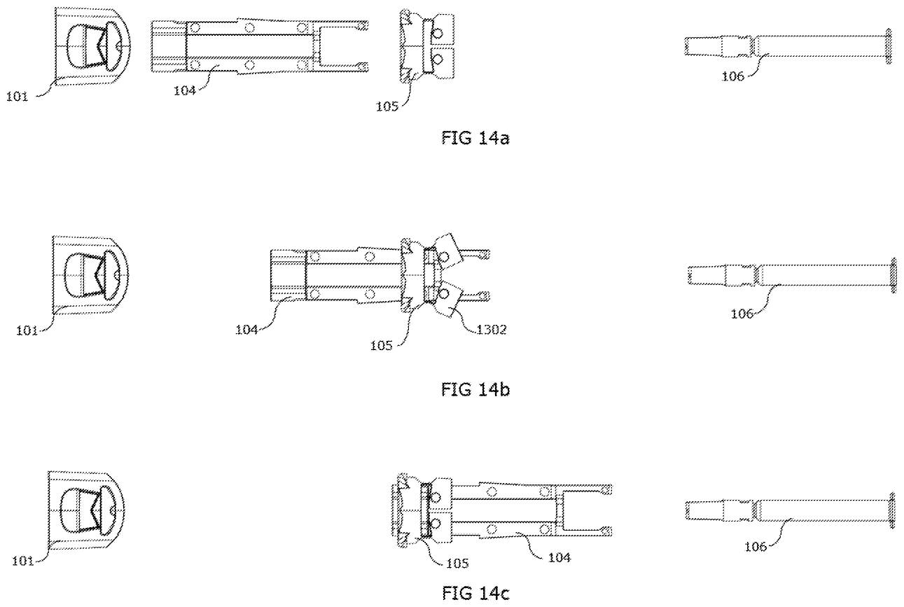

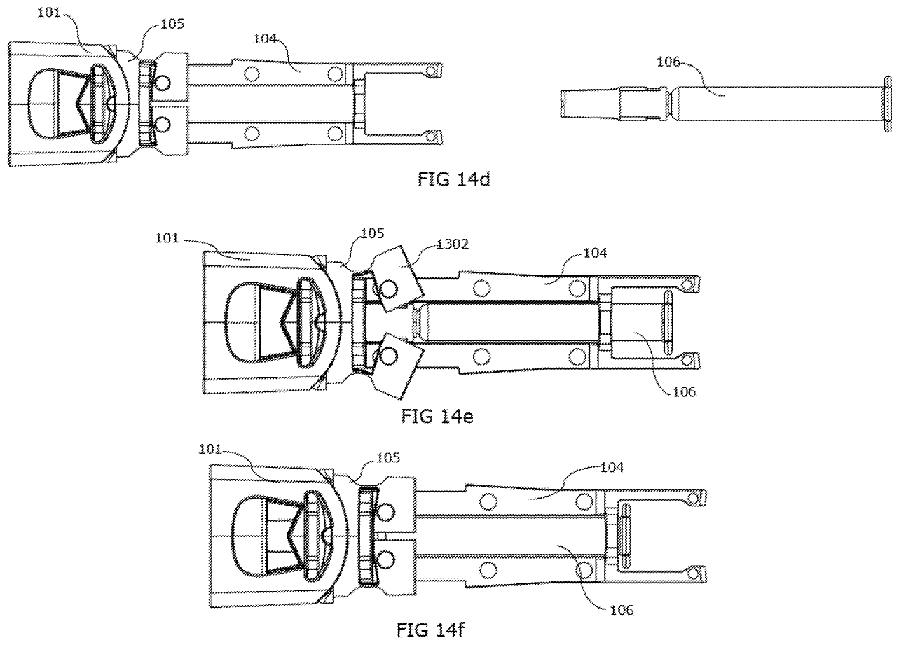

FIGS. 14a-f illustrate assembly of the syringe retention member 105 with the needle guard 104 and PFS 106. As shown in FIG. 14a, the cap 101, the syringe retention member 105, the needle guard 104 and the PFS 106 are initially separate.

During assembly of the autoinjector 100, the arms 1302 of the syringe retention member 105 are manually pivoted outwards and the needle guard 104 is inserted into an aperture (not visible) in the annular collar 1301 as shown in FIG. 14b. The arms 1302 may then be returned to their original position once the needle guard 104 is fully inserted, as shown in FIG. 14c.

Next, the cap 101 is placed against the syringe retention member 105 as shown in FIG. 14d. The opposing arms 1302 are then manually pivoted outwards as shown in FIG. 14e to allow the PFS 106 to be inserted within the syringe retention member 105 and needle guard 104, and the RNS of the PFS 106 is pushed into the cap 101. Once in position, the opposing arms 1302 are pivoted back to their original positions as shown in FIG. 14f.

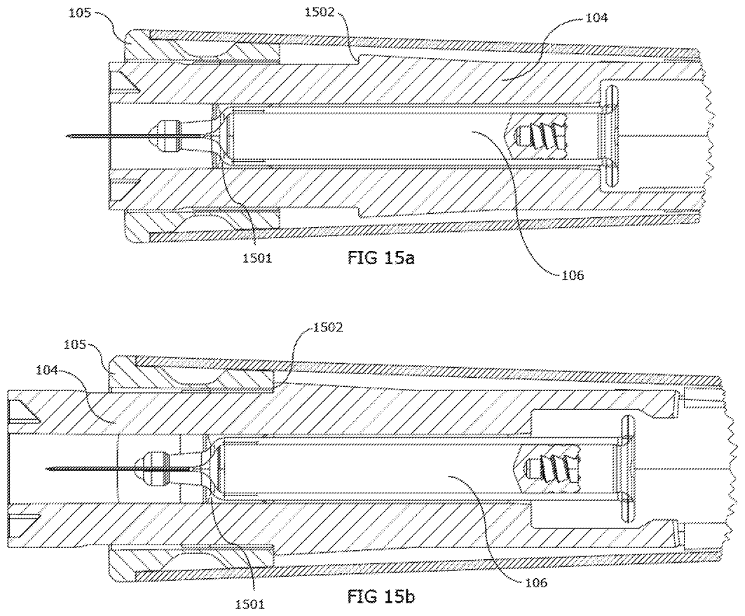

Moving on to FIGS. 15a and 15b, which show cutaway views of the syringe retention member 105, the PFS 106 is retained by shoulder sections 1501 protruding from the inner surfaces of the opposing arms 1302 distal of the pivot points 1303. The shoulder sections 1501 are shaped to engage with the shoulder of the PFS 106 in a circumferential gap between the RNS 805 and the barrel of the PFS 106. The shoulder sections 1501 provide a reaction force that opposes the longitudinal force exerted upon the PFS 106 by the bias spring 108 and prevents the PFS 106 coming out of the housing.

The opposing arms 1302 of the syringe retention member 105 are additionally shaped to abut against ledges 1502 in the outer surface of the needle guard 104. This abutment between the arms 1302 and the ledges 1502 prevents the needle guard 104 falling out of the housing 102.

Once assembled, the barrel of the PFS 106 provides a radially outward reaction force that prevents the opposing arms 1302 flexing inwards. This helps to retain the syringe retention member 105 in the housing 102 by preventing the locking elements 1304 from disengaging with the corresponding locking elements on the inner surface of the housing 102. The housing 102 acts to push the opposing arms 1302 inwards, thereby pulling all the components tightly together and securing the PFS 106 from movement. The syringe retention member may optionally be provided with a label wrapping to prevent flexing and provide evidence of tampering.

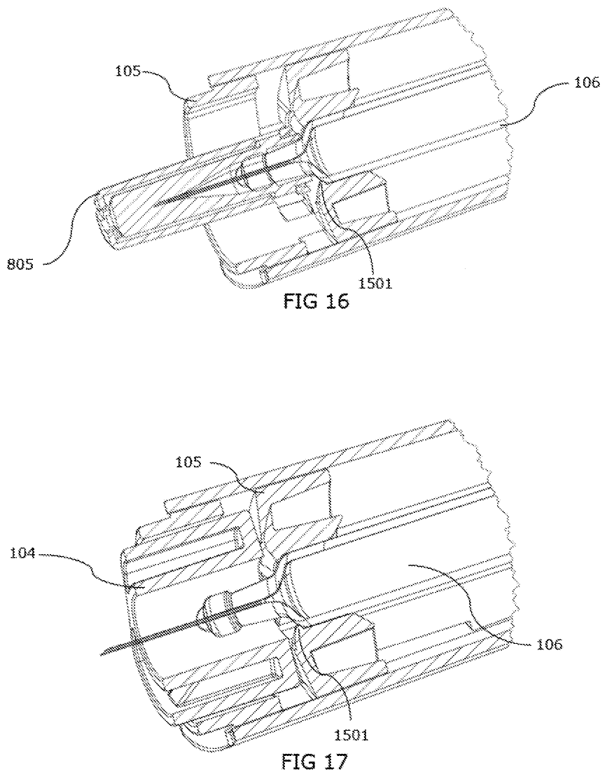

FIGS. 16 and 17 show alternative cutaway views of the syringe retention member 105 and PFS 106 with and without the needle guard 104 respectively.

While the illustrated syringe retention member 105 features two opposing arms 1302, alternatives are envisaged in which there are more arms, e.g. three, four or more opposing arms. When the number of opposing arms is odd, the arms will not directly oppose each other, but they will still oppose each other in the sense that are arranged on opposing sides of the syringe retention member in such a manner as to provide a net balancing force.

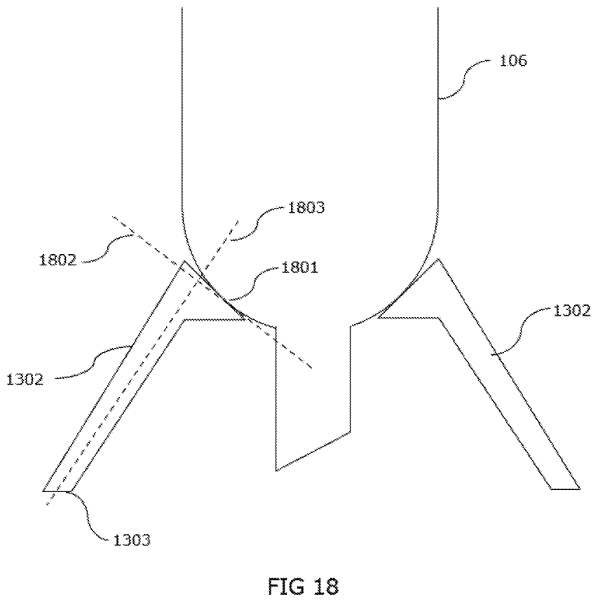

Having the shoulder sections distal of the pivot points creates a self-locking effect that acts upon the syringe: when a load is placed upon the syringe plunger, the force acting on the syringe abutment shoulder causes the arms to grip the syringe more tightly rather than prising them apart. To ensure that the support arms pivot radially inwards when a load is placed upon the syringe plunger, the engagement surface of each arm is preferably positioned proximally to a straight line extending through the pivot point each support arm perpendicular to a tangent of the syringe surface where the syringe abuts the arms. This relationship is illustrated in FIG. 18, which is a simplified illustration of the geometry of the arms 1302. As shown in FIG. 18, the PFS 106 abuts the left arm 1302 at point 1801. The tangent to the surface of the PFS 106 at point 1801 is illustrated by dashed line 1802. It can be seen that the point 1801 is proximal (and radially inwards) of a straight line 1803 through the pivot point 1303 perpendicular to the tangent 1802 (in other words, the point 1801 is at a proximal and radially inward position along the tangent 1802 relative to line 1803).

Although the syringe retention member 105 has been described in relation to an autoinjector, it could also be used to retain syringes in other drug delivery devices with or without a needle guard 104, such as an injector pen.

### Other Examples and Applications

It should be understood that the illustrated devices disclosed herein are merely exemplary, and the devices could potentially be provided with additional or fewer features whilst still falling within the scope of the appended claims. Likewise, the shapes and sizes of the components of the devices could differ from those illustrated.

In addition, unless specified otherwise, the order in which the method steps are presented is merely exemplary, and one skilled in the art will recognise that the steps of the methods disclosed herein could be performed in a different order (unless technically infeasible) and that additional or fewer steps could also be performed.

1. A drug delivery device comprising:
a housing;
a syringe received within the housing and comprising a plunger, wherein the plunger comprises a first guide surface arranged to abut a second guide surface within the housing at a non-perpendicular angle relative to a longitudinal axis of the plunger;
a drive element arranged to exert a depression force upon the plunger; and,
an activation element movable between a first position in which the activation element is arranged to prevent lateral movement of the plunger within the housing and a second position in which the activation element is arranged to allow lateral movement of at least part of the plunger within the housing,
wherein when the activation element is displaced from the first position to the second position, abutment between the first guide surface and the second guide surface causes a lateral displacement of the at least part of the plunger due to the depression force of the drive element, thereby releasing the plunger to move from an undepressed position to a depressed position under the depression force of the drive element.

2. The drug delivery device of claim 1, wherein the plunger comprises a first laterally protruding guide element comprising the first guide surface and wherein the second guide surface is on a second laterally protruding guide element.

3. The drug delivery device of claim 1, wherein the plunger comprises a laterally protruding guide element comprising the first guide surface and wherein the second guide surface is in a guide recess arranged to receive the laterally protruding guide element.

4. The drug delivery device of claim 1, wherein the second guide surface is on a laterally protruding guide element and wherein the plunger comprises a guide recess comprising the first guide surface arranged to receive the laterally protruding guide element.

5. The drug delivery device of claim 1, wherein the activation element is one or more of:
moveable in a longitudinal direction;
rotatable; and
moveable in a direction tangential to an outer surface of the plunger.

6. (canceled)

7. (canceled)

8. The drug delivery device of claim 1, wherein the activation element comprises an aperture shaped to receive a laterally protruding locking element of the plunger when the activation element is in the second position, and wherein the laterally protruding locking element abuts the activation element in the first position.

9. The drug delivery device of claim 1, wherein the plunger comprises a locking recess shaped to receive a laterally protruding locking element of the activation element when the activation element is in the second position, and wherein the laterally protruding locking element abuts the plunger in the first position.

10. The drug delivery device of claim 1, wherein the drive element is a compression spring.

11. (canceled)

12. The drug delivery device of claim 1, wherein the drive element is a piston.

13. The drug delivery device of claim 1, further comprising a moveable needle guard arranged to shield a needle coupled to a tip of the syringe, wherein the needle guard comprises the activation element.

14. The drug delivery device of claim 1, where the lateral displacement comprises bending and/or pivoting of the plunger.

15. (canceled)

16. A syringe retention member for retaining a syringe in a drug delivery device, comprising:
an annular collar; and,
one or more support arms extending longitudinally from a distal side of the annular collar, each of the one or more support arms pivotable relative to the collar about a respective pivot point,
wherein each of the one or more support arms comprises a shoulder section distal of the respective pivot point that provides a syringe abutment shoulder for engaging in a circumferential gap between a barrel of the syringe and a rigid needle shield of the syringe.

17. The syringe retention member of claim 16, wherein when a syringe is inserted into the syringe retention member an abutment surface of the syringe abuts a respective engagement surface of the syringe abutment shoulder of each respective support arm, and wherein the respective engagement surface is positioned proximally to a straight line extending through the respective pivot point of the respective support arm perpendicular to a tangent of the abutment surface where the respective engagement surface abuts the abutment surface.

18. The syringe retention member of claim 16, wherein the syringe abutment shoulder is formed by an inwardly-projecting syringe protrusion on each support arms.

19. The syringe retention member of claim 16, wherein an opening in the annular collar is shaped to receive a needle guard.

20. The syringe retention member of claim 16, wherein an outer surface of each of the support arms comprises a first locking element configured to interface with a corresponding second locking element on a housing or syringe carrier of the autoinjector so as to prevent removal of the syringe retention member from the housing.

21. The syringe retention member of claim 16, wherein when a syringe is received within the syringe retention member, a barrel of the syringe provides a radially outward reaction force that prevents the support arms from flexing inwards.

22. (canceled)

23. The syringe retention member of claim 16, wherein the one or more support arms comprises a pair of opposing arms.

24. (canceled)

25. An autoinjector cap comprising:
a cap housing; and,
a gripping plate received in an opening in a side of the cap housing and arranged to grip a rigid needle shield of a syringe retained within the autoinjector,
wherein the gripping plate comprises an aperture comprising a pair of opposing gripping surfaces configured to engage opposing sides of an outer surface of the rigid needle shield; and,
wherein the gripping plate is bent along a line bisecting the aperture such that each of the opposing gripping surfaces is angled towards a tip of the autoinjector cap.

26. An autoinjector comprising:
a housing;
a needle guard moveable between a retracted position and an extended position in which the needle guard is arranged to shield a needle of a syringe retained within the housing; and,
a biasing element arranged to bias the needle guard into the extended position,
wherein the needle guard comprises a first latch element arranged to interface with a corresponding second latch element fixed within the housing;
wherein the autoinjector further comprises an activation element initially in a first position in which the activation element is positioned to obstruct the second latch element and thereby prevent abutment between the first latch element and the second latch element; and,
wherein when the needle guard is moved from the extended position to the retracted position, abutment between the needle guard and the activation element causes the activation element to move from the first position to a second position in which the second latch element is exposed to the first latch element such that subsequent movement of the needle guard from the extended position to the retracted position is prevented by abutment between the first latch element and the second latch element.
